(19) United States
(12) Patent Application Publication
(10) Pub. No.: US 2003/0082625 A1
(43) Pub. Date: May 1, 2003

(54) Method for the pretreatment of a surface for the reduction of non-specific binding by chemical entities

(76) Inventors: Yong-Hee Lee, San Diego, CA (US); Kyoung-Jin Lee, San Diego, CA (US); Kevin Holme, San Diego, CA (US)

Correspondence Address: ARENT FOX KINTNER PLOTKIN & KAHN, 1050 CONNECTICUT AVENUE, N.W., SUITE 400, WASHINGTON, DC, 20036, US

(21) Appl. No.: 09/983,018

(22) Filed: Oct. 22, 2001

(51) Int. Cl. G01N 33/53; B08B 3/00; C12M 1/34; G01N 33/543

(52) U.S. Cl. 435/7.1; 436/518; 435/287.2; 134/26

(57) ABSTRACT

The present invention provides for a method for the pretreatment of a surface and/or reservoir, in particular a plastic surface preferably a reservoirs and more in particular filter membranes enclosed therein, for the reduction of non-specific binding by chemical entities, such as but not limited to small organic molecules with bodily fluids such as blood serum proteins, to said reservoirs, whereby the method makes use a non ionic surfactant or, a cationic surfactant or both kinds of surfactants. The invention also concerns kits for enabling the method described above.

### BACKGROUND OF THE INVENTION

The binding of small molecules and peptides to serum proteins is a very important parameter used in the pharmaceutical industry to measure potential effectiveness of a drug. When a molecule is highly bound to protein in the blood, the amount of drug available to diffuse into the target tissue is significantly reduced and the efficacy of the drug will inevitably be poor. Whether a small molecule binds to plasma protein or not usually depends on the size of the molecule, the amino acid composition and the tertiary structure of the molecule. When a small molecule binds to plasma protein, the interaction usually is a result of strong ionic and hydrophobic interactions.

Because blood contains several hundred proteins there is a high probability that any small molecule will exhibit some level of binding. Determining the level of binding, therefore, is critical and will directly correlate with in vivo efficacy of the molecule.

Predicting whether a molecule is going to show high or low protein binding based on molecular structure and other in silico methods has proven to be very difficult. One way to determine whether or not a molecule will exhibit high or low protein binding is to actually test the molecule directly in a protein-binding assay. This is a critical first step in characterizing the distribution of a small molecule with respect to the plasma compartment.

The most common method used to measure the level of protein binding is equilibrium dialysis. Many researchers use ultrafiltration centrifugal devices, containing size exclusion membranes to separate free from plasma bound drug.

The binding of small molecules however, to the plastics such as the polypropylene tubes and plates can be a problem. Polypropylene (PP) is currently considered the best type of commercially available plastic plate based on its low non-specific binding properties and solvent resistance. Even the non-specific binding to polypropylene can interfere with the calculation of accurate plasma protein binding values.

Recently Millipore introduced a polypropylene and Teflon mix plastic reservoir. However, also this material does not entirely eliminate the problem of non-specific binding and in particular hydrophobic and basic compounds will tend to bind the plastic but even more so the filter membranes often contained therein. The problem of non-specific binding of said compounds to these ultra filtration membranes has so far not been addressed sufficiently, in particular for pharmocodynamics assays where candidate compounds are measured for their ability to bind serum proteins or other biological molecules, such as target molecules.

Problems Solved by the Invention and General Concept of the Invention

The present invention provides for a method of pre-treating surfaces preferable plastic reservoirs and/or the membrane filters contained therein or provided separately thereby, reducing or eliminating the problem of non-specific binding of, in particular hydrophobic and basic compounds to said surfaces. The method is based on the observation that a pretreatment of said plastics and/or membranes with various surfactants optionally including the a step of equilibration will reduce the non-specific binding of various types of compounds. The invention makes use of non-ionic surfactants and cat-ionic surfactants for this purpose. Consequently, the present method for the pretreatment of a surface and/or a reservoir for the reduction of non-specific binding by chemical entities to said reservoir comprises the steps of, bedewing at least a portion of the reservoir with either a non ionic surfactant or, a cationic surfactant or, with both kinds of surfactants in sequential order. The invention is applicable in many technical fields such as but not limited to the field of filtration of chemical preferably biological elements, such as serum proteins combined with chemical compounds.

### DETAILED DESCRIPTION OF THE PREFERRED EMBODIMENTS

The inventors have found that the binding of small molecules to the plastics such as the polypropylene tubes and plates can be a problem. Polypropylene (PP) is currently considered the best type of commercially available plastic plate based on its low non-specific binding properties and solvent resistance. Recently Millipore introduced a polypropylene and Teflon mix plastic reservoir. However, also this material does not entirely eliminate the problem of non-specific binding and in particular hydrophobic and basic compounds will tend to bind the plastic but even more so the filter membranes often contained therein.

The inventors have made the observation that a pretreatment step of the respective plastic reservoir and/or the filter membrane contained therein with a non-ionic solvent or a cat-ionic solvent can reduce the non-specific binding of various kinds of organic compounds, in particular such compounds that are hydrophobic or basic in kind.

The present invention relates to a method for the pretreatment of a plastic surface for the reduction of non-specific binding by chemical entities to said surface which comprises the steps of, bedewing at least a portion of the reservoir with either a non ionic surfactant or, a cationic surfactant or, with both kinds of surfactants in sequential order. The method also relates to the pretreatment of a filter membrane wherein, the same steps are applied as outlined above.

In one embodiment the surface is part of a reservoir for holding or filtrating liquids and all or parts of the reservoir are bedewed.

In particular the invention concerns the pretreatment of a reservoir which is subdivided into at least two compartments whereby, the reservoir additionally comprises a filter membrane dividing said two compartments and, at least said membrane is bedewed with either the non-ionic surfactant or the cat-ionic surfactant. Various kinds of such reservoirs are being used in drug research the general structure of some of the embodiments of such reservoirs is depicted in FIG. 1. FIG. 1A depicts a reservoir with one chamber which is subdivided by a filter membrane into two sub-chambers. Here, the method according to the invention would ideally be performed in such a way that all portions of the upper sub-compartment are bedewed with the surfactant, and subsequently e.g. the serum and the compound are added, whereby the compound not bound to the serum proteins will flow past the filter membrane to the lower compartment. In an alternative setting this can be done using a reservoir which sits inside a tube that may placed into a centrifuge (FIG. 1.B). FIG. 1B shows another type of reservoir. In this embodiment of the method it may be best if all compartments are bedewed with the surfactant. It is essential that the compartment into which the compound to be analyzed is placed, is bedewed.

Ideally, the entire compartment is bedewed with the surfactant however, it may suffice to bedew the filter membrane part if, the plastic of which the reservoir is made is chosen from the group comprising plastics that have a reduced rate of non-specific binding, such as those which are composed of, e.g. 97.5% polypropylene and 2.5% Teflon.

The bedewing may be performed in various ways provided the parts get covered with surfactant that are desired to be covered. One may pipette the liquid into the reservoir or sink the reservoir into the surfactant. One may spray the surfactant onto the reservoir. In one embodiment the surface and/or reservoir is pretreated with the surfactant and then dried or dehydrated. It may then be stored in a dried state util it is needed.

The reservoir may actually be part of a larger array of reservoirs which are optionally bedewed as well. Such an array may have the shape or form of a microtiter plate. Well known microtitre plates are such plates that have, e.g. 96 wells or more. Such plates are known that have 96 wells wherein each well comprises a filter membrane. It may be advantageous in such a case to bedew all, some or few of the individual wells. It may also be advantageous to bedew some wells with a non-ionic surfactant and the other wells with a cat-ionic surfactant.

Ordinarily the filter membranes which are present in such filtration units are composed of regenerated cellulose (Millipore, UFC 3LGC00, 10 k NMWL) or polyethersulfone (Millipore, UFC 3BSC00, 10 k NMWL), however the invention is not limited to be applied to the filter or ultrafiltration membranes mentioned herein. In general membranes consisting essentially of microporous or ulfiltrafiltration from 3000 to 100,000 NMWL are well suited.

Filter membranes that can be used for the invention can be selected from but are not limited to:

a) Nylon which is usually uncharged hydrophilic base material whose surface is optionally modified by negatively charged groups, e.g., carboxyl- or hydroxyl groups;

b) Polyether sulfones which usually consists of uncharged hydrophilic base material whose surface is optionally modified by negatively charged groups, e.g., carboxyl- or hydroxyl groups;

c) Hydrophilically modified polyvinyldifluoride which usually consists of a hydrophobic base material that gains a hydrophilic character by surface modification;

d) Cellulose acetate which usually consists of an uncharged hydrophilic base material whose surface is optionally modified by negatively charged groups, e.g., carboxyl- or hydroxyl groups;

e) Cellulose mixed ester which usually consists of an uncharged hydrophilic base material whose surface is optionally modified by negatively charged groups, e.g., carboxyl- or hydroxyl groups or,

f) Regenerated cellulose which usually consists of an uncharged hydrophilic base material whose surface is optionally modified by negatively charged groups, e.g., carboxyl- or hydroxyl groups.

After bedewing the reservoir an equilibration or a centrifugation may be applied. Equilibration is ordinarily performed with a reagent chosen from the group comprising, phosphate buffered saline, saline, and Ringers solution.

The non ionic surfactant is chosen from the group comprising 0.01% to 20% polyoxyethylene sorbitan fatty acid esters (tween), polyoxyethylene alkyl ethers 0.01% to 20%, polyoxyethylene castor oil derivatives 0.01% to 20%, sorbitan fatty acid esters (span) 0.01% to 20%, poloxamer (pluronic) 0.01% to 20%, and glyceryl monooleate 0.01% to 20%.

The non ionic surfactant has been found to give very good results and is a preferred embodiment. Ideally between 0.01% and 20% is used, best results have been achieved using 5%.

The cationic surfactant is chosen form the group comprising 0.01% to 20% benzalkonium chloride, benzethonium chloride 0.01% to 20%, and cetrimide 0.01% to 20%.

The cationic surfactant benzalkonium chloride has been found to give very good results when used ideally between 0.01% to 20%. Best results have been achieved using 5%.

The combination of non ionic and cationic surfactants has been found to give poor results, but the combination of more than two surfactants from the same kind show good results.

The amount of liquid to be applied depends entirely on the surface area which is to be bedewed. Likewise one can envision that the reservoirs which optionally contain a filter membrane are stored in one of the surfactants.

Said non ionic surfactants or a cationic surfactants may be used for the pretreatment of a reservoir comprising a filter membrane, the filter membrane alone or only the reservoir. Also, the filter membrane may be pretreated before it becomes part of the reservoir.

In one embodiment of the invention the invention covers a kit for the analysis of biomolecules, wherein the kit comprises at least reservoir for said biomolecules, said reservoir optionally comprising a filter membrane for the filtrartion of said biomolecules, further comprising a non ionic surfactant preferentially in a reservoir or vial, or plastic tube or, a cationic surfactant preferentially in a reservoir or vial, or plastic tube, or with both surfactants in said containers. The surfactants may not be present in a premixed state but only separately. The analyses for which said kit may routinely by applied are such as the filtration of proteins of varying molecular weight in particular the filtration of proteins which have been inoculated previously with a organic or inorganic chemical substance. Such assays may be for example binding assays of chemical compounds to body fluids in particular serum proteins. These assays are common in pharmacological research where lead compounds are analyzed.

The invention further relates to plastic surfaces that have been pretreated according to the method according to the invention as well as a liquid holding reservoir, optionally comprising a filter membrane wherein, the reservoir is pretreated according to the invention.

### EXAMPLES

Various experiments were performed using the following reagents, surfactants and reservoirs (also comprising ultrafiltration units): human serum, pH 7.4 phosphate buffered saline (PBS), drug solution (i.e. compound) in serum at a concentration of 1-50 μM, and drug solution in PBS at a concentration of 1-50 μM were used as reagents. Surfactant solutions were 0.01-20% Tween 80 (polyoxyethylene 20 sorbitan monooleate) in PBS as neutral solution, 0.01-20% Benzalkonium chloride (BAK) in PBS as cationic solution, 0.01-20% Sodium lauryl sulfate (SLS) in PBS as anionic solution. An ultrafiltration reservoir (Millipore, Ultrafree-MC) comprising regenerated cellulose (UFC 3LGC00, 10 k NMWL) or polyethersulfone (UFC 3BSC00, 10 k NMWL) was used.

The upper ultrafiltration cup was connected to the reservoir tube and 25 μL surfactant solution was added to each well. After bedewing for 5 min, the reservoir tube containing the ultrafiltration cup was centrifuged for 10 min at 3000 g and room temperature.

200 μL of PBS was added to each ultrafiltration cup and equilibrated for 30 min. Remaining PBS was removed from the ultrafiltration cup by gentle tapping the cup. The upper ultrafiltration cup was reconnected to a new reservoir tube, and 400 μL drug solution (1-50 μM) in serum or PBS was applied and equilibrated for 1 hr at room temperature.

The ultrafiltration tube was centrifuged for 18 min at 3000 g at room temperature. 50 μL sample was taken from the receiver (filtrate) and donor (drug solution in serum or PBS) for bioanalysis. Non-specific binding was calculated as follows:

NSB=(C_{f1}-C_{f2})/C_{f1}, where NSB: non specific binding to membrane filter at pH 7.4 isotonic PBS, C_{f1}: free drug before filtration, and C_{f2}: free drug in the filtrate after filtration

### Example 1

Example 1 demonstrates the modulation of non specific binding (%) of drugs by 0.5% surfactant solution. Ultrafiltration reservoir (Millipore, Ultrafree-MC) comprising regenerated cellulose (UFC 3LGC00, 10 k NMWL) was used in example 1. 0.5% tween 80 or benzalkonium chloride (BAK) showed a significant decrease of non specific binding (NSB) for 10 drugs tested. The results of the modulation of non specific binding “NSB” (%) of drugs using a 0.5% surfactant solution are shown in FIG. 2.

Table 1 shows the modulation of NSB (%) of drugs by 0.5% surfactant solution.

TABLE 1

| Drugs | Control | Tween 80 | BAK |
|---|---|---|---|
| Antipyrine | 10 | 0 | 0 |
| Caffeine | 17 | 0 | 0 |
| Fluorocytosine | 2 | 0 | 0 |
| Theophylline | 8 | 0 | 0 |
| Ibuprofen | 29 | 2.3 | N/A |
| Ketoprofen | 34 | 4.1 | N/A |
| Propranolol | 91 | 46.5 | 13 |
| Etoposide | 91 | 23.1 | 28.3 |
| Hydrocortisone | 87 | 21.1 | 25 |
| Vinblastine | 95 | 95.2 | 64 |

Drug concentration: 5 uM
Filtration membrane: Regenerated cellulose
N/A: not applicable

### Example 2

Example 2 demonstrates the modulation of non specific binding (%) of drugs at various concentrations of surfactant solutions. Ultrafiltration reservoir (Millipore, Ultrafree-MC) comprising regenerated cellulose (UFC 3LGC00, 10 k NMWL) was used at example 2. An increase of surfactant concentration, e.g. of tween 80 or BAK showed a significant decrease of NSB for 4 tested drugs. FIG. 3 shows the results of the modulation of NSB (%) of drugs using various tween concentrations. Further, FIG. 4 shows the modulation of NSB (%) of drugs by applying the method according to the invention whereby the surfactant is benzalkonium chloride “BAK” at various concentrations.

Table 2 shows the modulation of NSB (%) of drugs by various concentrations of tween 80 and benzalkonium chloride (BAK) surfactant solutions.

TABLE 2

| Drugs | 0.5% Tween | 1% Tween | 2% Tween | 5% Tween |
|---|---|---|---|---|
| Propranolol (5 uM) | 66 | 65 | 60 | 52 |
| Hydrocortisone (5 uM) | 52 | 35 | 33 | 30 |
| Drugs | 0.5% BAK | 1% BAK | 2% BAK | 5% BAK |
| Propranolol (10 uM) | 14 | 15 | 5 | 10 |
| Hydrocortisone | 30 | 22 | 16 | 14 |
| (10 uM) | | | | |
| Verapamil (10 uM) | 43 | 34 | 25 | 12 |
| Vinblastine (10 uM) | 82 | 79 | 68 | 59 |

Filtration membrane: Regenerated cellulose

### Example 3

Example 3 demonstrates a negligible modulation of NSB (%) of drugs by human serum or 0.5% sodium lauryl sulfate (SLS) as can be seen also from Table 3 shown below. Ultrafiltration reservoir (Millipore, Ultrafree-MC) comprising regenerated cellulose (UFC 3LGC00, 10 k NMWL) was used at example 3.

Table 3 shows a negligible modulation of NSB (%) of drugs by human serum or 0.5% sodium lauryl sulfate (SLS).

TABLE 3

| Markers | Control | Serum | SLS |
|---|---|---|---|
| Propranolol | 91 | 90 | 84 |
| Etoposide | 91 | 93 | N/A |
| Hydrocortisone | 87 | 84 | 62 |

Drug concentration: 50 uM
Filtration membrane: Regenerated cellulose
N/A: not applicable

### Example 4

Example 4 relates to NSB (%) of drugs on various filtration membranes. Note, that regenerated cellulose (RC) and polyether sulfone (PES) showed a high NSB for 4 tested drugs.

Table 4 shows NSB (%) of drugs on various filtration membranes.

TABLE 4

| Markers | RC | PES |
|---|---|---|
| Propranolol | 91 | 69 |
| Etoposide | 91 | 93 |
| Hydrocortisone | 87 | 71 |
| Vinblastine | 95 | 95 |

Drug concentration: 10 uM
Filtration membrane:
RC: Regenerated cellulose
PES: Polyethersulfone

### FIGURE CAPTIONS

FIG. 1:

FIG. 1 shows a typical application of the method according to the invention. IN this set-up the aim is to reduce the non-specific binding of test compound to the reservoirs. FIG. A shows the filtration unit. FIG. B shows the filtration unit plus reservoir. This unit fits into a centrifuge.

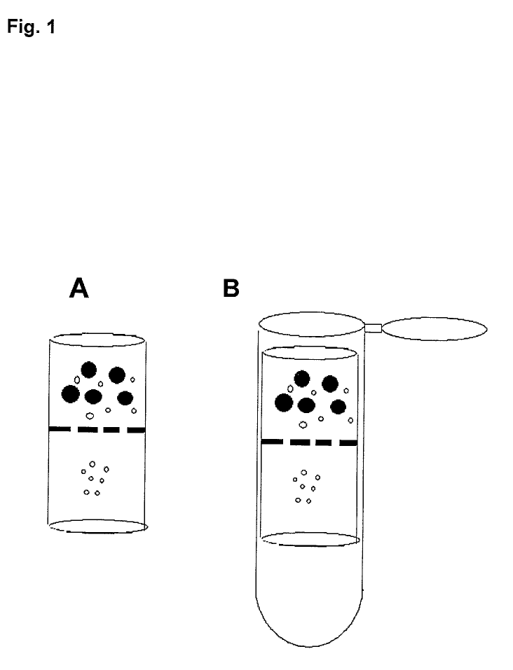

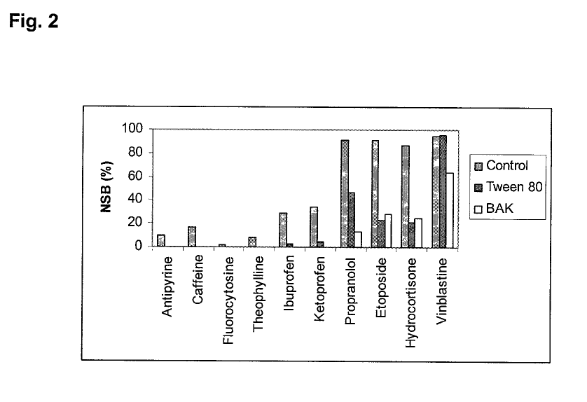

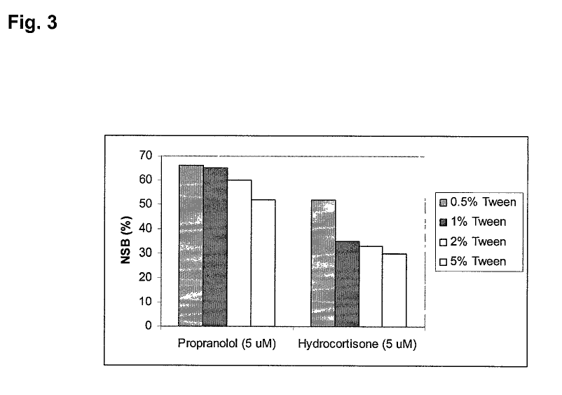

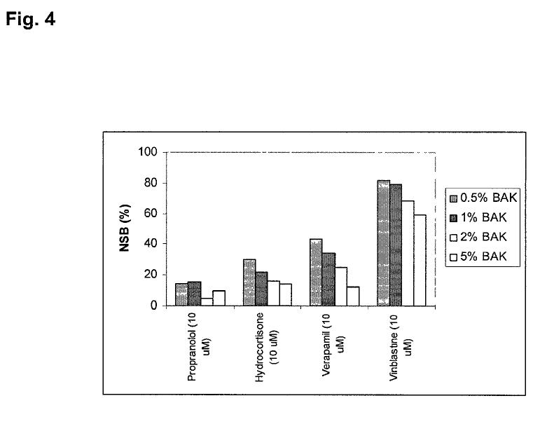

The effect of non specific binding of test compounds to the reservoir and/or the ultrafiltration unit is detrimental to the determination of the amount of test compound bound to the serum proteins. Under ordinary circumstances the amount of test compound would be determined by measuring the amount of test compound that passes the ultrafiltration unit. However, without making use of the present invention this figure may be incorrect as much test compound may be bound non specifically by the reservoir and thus not male its way to the lower reservoir.

Making use of the present invention, the plastic surface that is bedewed is the reservoir that holds the serum and compound to be tested for binding to the serum proteins before it is filtrated through the ultrafiltration membrane. Note, that also the filter is bedewed with surfactant. In an alternative setting the entire device is bedewed or dipped into the surfactant. Thus, non serum binding test compound amount can be accurately determined because non-specific binding to the reservoir was reduced.

FIG. 2:

0.5% tween 80 or benzalkonium chloride (BAK) showed a significant decrease of non-specific binding (NSB) for 10 tested drugs as can be seen by the graphical representation in FIG. 2.

FIG. 3:

FIG. 3 shows the modulation of NSB (%) of drugs by applying the method according to the invention whereby the surfactant is tween 80 at various concentrations.

FIG. 4:

FIG. 4 shows the modulation of NSB (%) of drugs by applying the method according to the invention whereby the surfactant is benzalkonium chloride “BAK” at various concentrations.

1. Method for the pretreatment of a plastic surface for the reduction of non-specific binding by chemical entities to said surface comprising the steps of,
bedewing at least a portion of the surface with either
a) a non ionic surfactant or,
b) a cationic surfactant or,
c) with both kinds of surfactants in sequential order.

2. Method according to claim 1 wherein,
the surface is part of a reservoir for holding or filtrating liquids and all or parts of the reservoir are bedewed.

3. Method according to claim 1 or 2 wherein,
reservoir is subdivided into at least two compartments, the reservoir additionally comprises a filter membrane dividing said two compartments

4. Method according to claims 1 to 3 wherein,
after bedewing the reservoir an equilibration step is performed.

5. Method according to claim 3 wherein,
the equilibration step is performed with a reagent chosen from the group comprising, phosphate buffered saline, saline, and Ringers solution.

6. Method according to any of the above claims wherein,
the non ionic surfactant is chosen from the group comprising 0.01% to 20% polyoxyethylene sorbitan fatty acid esters (tween), polyoxyethylene alkyl ethers, polyoxyethylene castor oil derivatives, sorbitan fatty acid esters (span), poloxamer (pluronic), and glyceryl monooleate.

7. Method according to claims 1 to 6 wherein,
the cationic surfactant is chosen form the group comprising 0.01% to 20% benzalkonium chloride, benzethonium chloride, and cetrimide.

8. Use of a non ionic surfactant or a cationic surfactant for the pretreatment of a plastic reservoir comprising a filter membrane.

9. Use of a non ionic surfactant or a cationic surfactant according to claim 8 wherein, the filter membrane is chosen from the group comprising regenerated cellulose, and polyethlsulfone.

10. Use of a non ionic surfactant according to claim 8 or 9 wherein, the non ionic surfactant is chosen from the group comprising 0.01% to 20% polyoxyethylene sorbitan fatty acid esters (tween), polyoxyethylene alkyl ethers, polyoxyethylene castor oil derivatives, sorbitan fatty acid esters (span), poloxamer (pluronic), and glyceryl monooleate.

11. Use of a cationic surfactant according to claim 8 or 9 wherein, the cationic surfactant is chosen from the group comprising 0.01% to 20% benzalkonium chloride, benzethonium chloride, and cetrimide.

12. Kit for the measurement of binding of small molecules or peptides to serum proteins comprising,
a) a reservoir comprising at least two compartments, the reservoir additionally comprising a filter membrane dividing said two compartments and,
b) either a cationic surfactant or a nonionic surfactant or both.

13. Plastic surface that has been pretreated according to any of claims 1 to 7.

14. Liquid holding reservoir, optionally comprising a filter membrane wherein, the reservoir is pretreated according to any of claims 1 to 7.
